(19) United States
(12) Patent Application Publication
(10) Pub. No.: US 2012/0195958 A1
(43) Pub. Date: Aug. 2, 2012

(54) Implantable devices

(75) Inventors: Timothy O'Brien, Galway (IE); Udo Greiser, Galway (IE); Faisal Sharif, Co. Galway (IE); Sean Hynes, Co. Galway (IE); Jim Crowley, Co. Galway (IE)

(73) Assignee: NATIONAL UNIVERSITY OF IRELAND, GALWAY, Galway (IE)

(21) Appl. No.: 13/256,262

(22) PCT Filed: Mar. 26, 2010
(86) PCT No.: PCT/EP2010/054052

§ 371 Date: Apr. 19, 2012

(30) Foreign Application Priority Data
Mar. 27, 2009 (IE) 2009/0236

(51) Int. Cl. A61K 9/127; A61M 27/00; B05D 5/00; A61K 31/7088

(52) U.S. Cl. 424/450; 514/44 R; 604/544; 427/2.1; 427/2.25

(57) ABSTRACT

The present invention relates to implantable devices, which are coated or coatable with liposomes. The present invention further relates to liposomes encapsulating nucleic acids termed lipoplexes, to methods for the manufacture and coating of such implantable devices, and to the use of such implantable devices to improve treatment of patients with coronary artery disease or other vascular diseases and cancers.

### FIELD OF THE INVENTION

The present invention relates to implantable devices, which are coated with liposomes. In particular the invention relates to stents, which are coated with liposomes. The present invention further relates to liposomes encapsulating nucleic acids termed lipoplexes, to methods for the manufacture and coating of such implantable devices, and to the use of such implantable devices to improve treatment of patients with coronary artery disease or other vascular diseases and cancers.

### BACKGROUND TO THE INVENTION

An escalating occurrence in heart, neurological, urinary and orthopaedic disorders in the general population, has prompted an increase in research, aiming to improve the safety and effectiveness of implantable medical devices. Numerous devices have been developed across a variety of fields, examples of which include, coronary/peripheral artery stents, intravenous canulae, urinary catheters, implantable coronary devices, bone plates, bone prostheses, dental implants, blood vessel prostheses, artificial heart valves, skin repair devices, contact lenses. The device may be intended as a temporary device as with intravenous canulae or as a permanently implanted device such as a heart valve or bone prostheses. Despite being as inert as possible once implanted and exposed to human tissue, implantable devices whether temporary or permanent, are treated as a foreign entity by the human body. Consequently, such devices elicit an immune and or fibrotic response, which may have dire implications for their longevity. Furthermore, implantable devices are prone to infection and may therefore, become a focus for sepsis, which at the very least requires their premature removal, if not causing a life-threatening situation.

Coronary artery disease is a leading cause of death and disease in the developed world and is rapidly increasing in the developing world. Cardiovascular disease (CVD) contributes to more than 50% of all deaths in Europe and it is estimated that 1 in 4 US residents has CVD in one form or another. The development of focal atherosclerotic lesions in the coronary artery gives rise to hypoperfusion of the heart resulting in a lack of oxygen for the cardiac muscle and subsequently ischemic heart disease or in more severe cases a myocardial infarction. In the advanced stages, plaque disruption or erosion of the atherosclerotic lesion leads to acute arterial thrombosis that causes unstable angina, myocardial infarction, sudden cardiovascular death and stroke, which remains one of the leading causes of morbidity and mortality worldwide. Similarly, vein graft failure occurs in coronary artery bypass grafts.

At present, a popular treatment option for stable coronary artery disease is pharmacotherapy. Disease progression and the identification of unstable focal lesions in the vasculature of the heart using angiography however, is an indication for the use of percutaneous transluminal coronary angioplasty followed by stenting, to prevent blockage of the vessel's lumen. Angioplasty and stenting are popular treatment options due to their favourable acute results, as well as improving long-term clinical outcomes in native coronary and graft diseases. Stents are metal scaffolds, which provide mechanical support to the arterial wall and their aim is to maintain the vessel's diameter following dilation of an endovascular balloon at the site of a focal lesion. However, when using a bare metal stent, up to 30% of these may fail due to the proliferation of smooth muscle cells in the vessel wall in response to the injury caused by the balloon inflation. This is termed in-stent restenosis. In addition, balloon-dilation results in the removal of the protective endothelial layer on the luminal surface of the vessel, which normally acts to prevent thrombosis occurring. Hence patients who have had a stent deployed must adhere to a course of anti-clotting therapy for six months to a year. In addition recent evidence has been published regarding the risk of late stent thrombosis up to 68 months after stent deployment (Balbi et al. 2010, Nagrani et al., 2010). This emphasises the need to heal the endothelial layer of the vessel wall after stent deployment.

At present strategies for delivering therapeutic genes to the vessel wall to improve in-stent restenosis include instilling them through catheters at the time of angioplasty, which has not resulted in significant improvement in restenosis (Laitinen et al. 2000; Hedman et al. 2003). Stents represent an ideal platform for localized gene delivery, acting as reservoirs for vectors allowing prolonged and localized release without systemic side effects.

Drug eluting stents (DES) are now routinely used for occlusive atherosclerotic coronary lesions to contain the problem of restenosis. However, animal studies have shown that DES can cause local toxicity to the vessel wall in the form of medial necrosis, intimal proliferation, chronic inflammation and delayed re-endothelialization of the stents. Drug-eluting stents attempt to prevent in-stent restenosis using pharmacological agents delivered from the surface of a stent to the local vessel wall. The agent acts to reduce inflammation and prevent smooth muscle cell proliferation. However, despite the ever increasing use of drug-eluting stents for coronary stenting along with periprocedural and long-term administration of anti-clotting agents, post procedural restenosis remains a significant problem (10-15% in high risk patients). There is also a risk of thrombosis occurring subsequent to the discontinuation of anti-clotting therapy since drug-eluting stents also inhibit the protective endothelial layer.

Similar problems also exist when stenting in the peripheral vasculature, in particular the superficial femoral artery as well as other vascular beds. Patients suffering from peripheral vascular disease exit in a spectrum ranging from focal stenosis with intermittent claudication causing pain and immobility to critical ischemia with complete occlusions of the vessel. This type of disease process leads to tissue death, necrosis, life-threatening gangrene with amputation as the only course of treatment. Significant difficulties with restenosis occur in peripheral stenting. Therefore, balloon angioplasty remains a popular physician choice to treat patients with these lesions.

Gene eluting stents provide an alternative treatment strategy for the prevention of thrombosis and restenosis through enhanced re-endothelialisation. Similar to DES, these stents deliver local bioactive agent to the vasculature in combination with revascularization procedure. The eluting bioactive agent in the case of a gene eluting stent is a suitable vector encoding a therapeutic gene. A multitude of vector systems have been evaluated for the introduction of genes into the vascular tissues. These include both viral and non-viral (plasmid/liposome) vectors. The safety and feasibility of viral mediated stent based delivery of the reporter genes to the blood vessel wall has been previously documented (Sharif F et al., 2006), which shows that viral vectors especially adenovirus and AAV serotype 2 can be delivered locally to the blood vessel wall over a prolonged duration using PC-stent technology. In addition successful transduction of rabbit iliac arteries with therapeutic eNOS gene, using adenovirus mediated gene eluting stents resulting in enhanced re-endothelialization with a reduction in intimal hyperplasia at four weeks post gene delivery has been observed (Sharif et al, 2008). For vascular gene therapy, viral vectors have received more attention because of their larger insert size and their innate ability to infect most vascular cells (French B A et al., 1994). Although most recombinant viral vectors are modified to minimize immune and/or inflammatory response (Gerzten R E et al., 1996; Schulick A H et al., 1997), their advancement to the clinics has been slowed by persistent safety concerns. An ideal vector is characterized by its high efficiency, cell specificity, low toxicity, unlimited insert size, prolonged expression and lack of immunogenicity. Non-viral vector based gene transfer could have significant advantages, especially because of their enhanced safety profile as compared to viral vectors. However, like viral vectors, naked DNA delivery in vivo induces an immune host response. This results in low levels of transfection efficiency and short term gene expression. In order to circumvent this, plasmids have been mixed with cationic lipids in water to form hollow spheres, known as liposomes.

Liposome-mediated gene delivery represents an interesting alternative to viral gene delivery as they are inexpensive, contain a large insert size and have less biosafety concerns. Liposomal structure is modifiable to achieve target specific liposomes, especially by the addition of receptor specific antibodies. However, to date, the level of transfection achieved with these vectors is low. Several early studies of direct in vivo vascular gene delivery using plasmid DNA, in complex with liposome-based carriers achieved a low level of transduction with these vectors (0.1-1%), even with modification of lipid composition (5% transduction of target cells) (Mazur et al., 1994, Flugelmann M Y et al., 1992). However, despite a better relative biosafety profile compared with viral vectors the systemic delivery of liposomes complexed with plasmid is not without its problems. Systemic delivery of liposomes may result in the induction of an inflammatory/complement response once it comes into contact with the reticuloendothelial system (Li and Szoka, 2007). Furthermore, there is a high rate of first pass elimination by endothelial cells in the lung and shortly after by Kuppfer cells in the liver. This is thought to be due to systemic interactions of cationic lipids with serum proteins and opsinins. A lack of the tissue tropism observed with viral gene delivery vectors makes it difficult for liposomes to target diseased tissue. Attempts have been made to circumvent these issues using polymer coatings to protect the liposome such as polyethylene glycol (PEG) and constructing it so that under a particular pH, as can occur in inflamed tissue, the PEG covering detaches and allows the liposome to target particular tissue conditions (Torchilin et al, 2006). The current invention circumvents these systemic considerations by using a stent-based platform to deliver the liposomes directly to the wall of a blood vessel.

Plasmid-based delivery from coated-stents has low efficiency. A study by Takahasi et al. (2003) could not demonstrate LacZ expression histochemically in a blood vessel wall using a plasmid from a stent indicating very low level of transfection. A plasmid encoding a reporter gene has also been delivered to the blood vessel wall from a coated stent resulting in a transfection efficiency of approximately 1% (Klugherz et al., 2000) which was subsequently modified by adding denatured collagen with an increase in cellular expression of reporter gene to 10% (Perlstein et al., 2003). Adenoviral and adeno-associated viral delivery from a stent has been used (Sharif et al 2006). In addition, tethering mechanisms to bind adenovirus to the stent coating by have also been described (Klugherz et al., 2002; Fishbein et al., 2006, Fishbein et al., 2008). This method achieved a transfection efficiency of 5.9% in pig coronary arteries at 7 days post-stent deployment. Thus far, the delivery of therapeutic genes has been through the use of either inefficient plasmid-based systems (Walter et al. 2004) or using adenoviral-based systems which also have relatively low expression (2%) and associated biosafety concerns (Johnson et al., 2005.

The present inventors have surprisingly found highly efficient expression of genes from a liposome-coated stent. The nucleic acid-liposome complex is designated herein, as a “lipoplex”. Whilst not wishing to be bound by any particular theory, it is believed that high level gene expression depends on a stable interaction of the lipoplexes and the bare metal stent or the PC coated stent, and the lipoplexes protection of plasmid DNA against degradation, an optimized release rate of the transfecting agent from the stent and high transfection efficiencies of predominantly macrophages a heretofore undescribed phenomenon discovered by the inventors. Cationic lipids have an amphipathic character with the hydrocarbon tail of the molecule being hydrophobic and its polar head being hydrophilic. When suspended in an aqueous environment the cationic lipid adopts various structural phases, including micellar, lamellar, and inverted hexagonal phase. These specific properties allow the cationic lipids to to form liposomes. It is well known that liposomes are microscopic vesicles comprising a lipid bilayer. Lipid bilayers occur in aqueous solution when hydrophobic tails line up against one another, forming a membrane with hydrophilic heads on both sides facing the aqueous phase.

The lipoplex formulation of choice of the invention is based on optimized ionic interactions between negatively charged plasmid molecules and positively charged lipids such as cationic lipids, for high complex stability. The cationic lipid mainly serves as a “binding partner” for the plasmid DNA (via ionic charges), forming the “lipoplex”. The cationic lipid also potentially acts as a condensing agent making the plasmid molecule more compact facilitating higher transfection efficiency.

The addition of the “helper lipid” lipid facilitates uptake of the lipoplexes by the cells and stabilizes the lipoplex structure. The helper lipid binds the cationic lipid, via non-ionic interactions, which is energetically favoured, as a low amount of energy is needed for this interaction. It is believed that the helper lipid improves transfection efficiencies due to their unique properties and ability to alter the phase of the lipoplex facilitating entry into the cells.

The current inventors have found that the addition of the helper lipid 1, 2-dioleoylphosphatidylcholine (DOPE) to the lipoplex enables in vivo transfection rates of local tissues including macrophages that are close to optimised in vitro transduction rates. Lipoplex formulations composed of helper lipids, such as DOPE, are in liquid crystalline phase and it is this state of the lipid bilayer that makes the lipoplex particle fusogenic with respect to cellular uptake or “adherent” to the cellular surface. Lipoplex formulations composed of helper lipids, such as cholesterol, are in gel phase. Lipoplexes in gel phase, are taken up by cells in vitro to a lesser extent than those lipoplexes in the liquid crystalline phase. This scenario may, however, not apply to an in vivo setting where other factors such as complex stability under shear stress, circulation times, bioavailability, tissues specificity and immunogenic properties of the lipoplexes play more important roles.

It is believed that neointima grows towards the lipo-stent and are readily transfected by stent associated lipoplexes upon contact. It has been observed that lipoplexes generally result in a higher transfection efficiency of rapidly dividing cells than in resting, confluent cell cultures. Successful gene delivery from a stent platform will also take advantage of rapid and efficient uptake of cationic lipoplexes by macrophages while delivering genes such as IL-10 that transform macrophages from an inflammatory state into a non-inflammatory state. In parallel, eNOS delivered from stents can suppress smooth muscle proliferation and initiate re-endothelialisation. The inventors found in the current inventive approach that the local levels of nitric oxide (NO), a freely diffusible compound, produced by eNOS-lipofected macrophages or smooth muscle cells, are elevated and sufficiently high enough to induce stimulation of re-endothelialisation.

The efficacy observed with respect to re-endothelialisation of the injured blood vessel after stenting, the high transfection efficiencies and long-term gene expressions make the cationic lipoplexes described in this invention ideal implantable device based transfection agents. In particular, the lipoplexes are ideal transfection agents of cells, such as dividing smooth muscle cells and macrophages that invade areas occupied by a stent, and a preferred means of prevention of in-stent restenosis caused by proliferating smooth muscle cells. Altering the body's interaction with implantable medical devices such as stents, in this way is of immense benefit to the host. In particular, modifying the cellular or immune response could serve to provide a solution to the problems associated with implantable devices and prevent device failure whilst maintaining an effective immune reaction to infection using gene modulation through effective gene delivery.

### OBJECT OF THE INVENTION

It is an object of the invention to provide an implantable medical device which has been coated in vitro or in situ with various coatings to deliver liposome-mediated genes to the local areas which may also be done in conjunction with a drug eluting stent. The device may be in particular a stent which delivers genes to a blood vessel wall. The use of various potential therapeutic genes in the stents of the invention could prevent in-stent restenosis through anti-inflammatory effects on macrophages, inhibition of smooth muscle proliferation as well as enhancing endothelial regeneration, which could help prevent late thrombosis subsequent to anti-clotting therapy and reduces the need for anti-clotting therapy. This may be achieved in a peripheral vascular bed also.

It is also an object to provide stents for use in the treatment of vein graft failure occurring in coronary artery bypass grafts. Liposome-mediated gene elution from a stent could result in the prevention of neointimal hyperplasia and failure of the graft.

### SUMMARY OF THE INVENTION

According to the present invention there is provided an implantable device either coated prior to implantation or coated in situ with a nucleic acid-encapsulating liposome or lipoplex.

In situ methods of coating implantable devises such as stents are known from Pfeiffer et al, Liu et al and Nikol et al.

The nucleic acid sequence is preferably located on a plasmid.

The implantable device may be a stent, in particular a coronary or peripheral artery stent, intravenous canulae, urinary catheters, implantable coronary devices, cardiac patches, bone plates, bone prostheses, bone patches, dental implants, blood vessel prostheses, artificial heart valves, skin repair devices, heart patches, bone patches, contact lenses. The implantable device may be intended as a temporary device or as a permanently implanted device.

The nucleic acid-encapsulating liposome or lipoplexmay be based on mixture of at least one cationic lipid or a cationic polymer and at least one other lipid called a “helper lipid”. “Helper lipids” serve to improve cationic lipid mediated transfection efficiency

In a still preferred embodiment of the current invention the cationic lipid may be selected from the group comprising (N-[1-(2,3-dioleyloxy)propyl]-n,n,n-trimethylammonium chloride (DOTMA) and dimethyldioctadecylammonium bromide salt (DDAB, N-(1-(2,3-Dioleoyloxy)propyl)-N,N,N-trimethylammonium methylsulfate (DOTAP) and 1,2-Dioleoyl-3-Trimethylammonium-Propane (chloride salt) (DMRIE). The helper lipid may be selected from the group comprising cholesterol (CHOL), 1-Palmitoyl-2-Oleoyl-sn-Glycero-3-Phosphocholine (POPC) and dioleoylphosphatidylethanolamine (DOPE) or any derivatives of gangliosides, sphingosine, sphingomyelin, prostaglandins, arachidonic acid, synthetic polymers or synthetic compounds suitable of forming particles capable of mammalian cell transfection.

In a preferred embodiment the nucleic acid-encapsulating liposome or lipoplex of the current invention comprise the following formulations:

(a) The mixture of cationic lipid DDAB and the helper lipid DOPE, form the lipoplex termed hereafter as Lipoplex 1 (Lx1) or,
(b) The mixture of cationic lipid DDAB and the helper lipids Chol/POPC, form the lipoplex termed hereafter as Lipoplex 2(Lx2) or,
(c) The mixture of cationic lipid DOTMA and the helper lipid DOPE, form the lipoplex termed hereafter as Lipoplex 3 (Lx3).

Surprisingly the inventors have found that Lipoplex 2 obtained after the addition of reporter gene plasmid DNA or therapeutic gene plasmid DNA (eNOS) to pre-formed liposomal formulations composed of cholesterol (CHOL), 1-Palmitoyl-2-Oleoyl-sn-Glycero-3-Phosphocholine (POPC) and DDAB resulted in long term gene expression and high in vivo transfection efficiencies of cells and tissues in a stented area. Similar results were obtained for Lipoplex 1 i.e. lipoplexes composed of DDAB, DOPE and plasmid DNA.

In a suitable embodiment of the current invention the amount of cationic lipids and the amount of helper lipids may vary from a 1:1 to a 1:5 molar ratio. The ratio of cationic lipid amount (nmole) to nucleic acid/plasmid amount (μg) may vary from approximately 3:1 to 6:1.

[symbol]

Some important genes which may be encapsulated by the liposome of the current invention include NOS (nitric oxide synthase) genes, iNOS (inducible NOS), eNOS (endothelial NOS), nNOS (neuronal NOS) and a phosphomimetic NOS, VEGF (vascular endothelial growth factor), (Tissue inhibitor of metalloproteinase) TIMP I-III, protein kinase G, prostacyclin synthase gene, SOD (superoxide dismutase) and GAX (an anti-proliferative homeobox gene). eNOS overexpression could prevent stent failure stenosis by preventing smooth muscle proliferation and enhancing endothelial regeneration.

Alternatively, siRNA, miRNA, or shRNA encapsulated by the liposome of the current invention and coated onto stents, in particular PC stents, bare metal stents, or another implantable device, may suppress genes that downregulate eNOS activity or participate in negative regulation of eNOS activity (e.g. C-reactive protein, angiotensin II AT I receptor, PPAR gamma, HSP70 and sONE). Additionally, eNOS siRNA encapsulated by DOTMA/DOPE liposomes and coated onto a stent, in particular PC stents or bare metal stents, could be used to suppress the role eNOS plays in enhancing tumor formation.

Nitric oxide synthase (NOS) genes may be encapsulated by liposomes of the current invention and coated onto stents, in particular PC stents or bare metal stents, for delivery to the vasculature which will allow their efficient expression. The expressed NOS genes may then produce nitric oxide (NO) in situ from an L-arginine substrate. The level of NO produced will depend on the isoform of NOS encapsulated. The endothelial isoform (eNOS) will produce a constitutive amount of nitric oxide (NO) and is calcium dependent, a phosphomimetic form of eNOS will produce an increased amount of NO in a calcium independent manner.

Alternatively, oligo nucleotides, siRNA, miRNA, or shRNA encapsulated by liposomes of the current invention and coated onto stents, in particular PC stents, bare metal stents, or another implantable device may suppress genes that downregulate eNOS activity or participate in negative regulation of eNOS activity (e.g. C-reactive protein, angiotensin II AT I receptor, PPAR gamma, HSP70 and sONE).

Nitric oxide has been shown to produce two effects in the vasculature; first it prevents smooth muscle cell proliferation through apoptotic dependent and independent mechanisms. It can also enhance regeneration of an endothelial cell layer where this has been removed/damaged in a blood vessel. This combination of contrasting effects on endothelial cells and the underlying smooth muscle cells would have applications in preventing coronary artery disease. The proliferation of smooth muscle cells post stent deployment is a significant factor in occluding a stent and causing it to fail. In addition, the removal of the endothelial cell layer through balloon deployment of the stent reveals an underlying prothrombotic extracellular matrix and surface. Thrombus formation on this surface is also a significant cause of stent failure due to occlusion. Overexpression of eNOS and the subsequent increase in NO bioavailability to the vessel would prevent stent failure by enhancing endothelial regeneration thereby preventing thrombosis and subsequent stent occlusion.

The aim of stenting is to maintain the patency of cardiac vessels and thereby maintaining cardiac perfusion. In contrast in neoplastic tissue it would be of therapeutic benefit if vascular perfusion could be decreased through anti-angiogenic strategies. It is thought that eNOS plays an enhancing role in established tumours through angiogenic pathways providing vascular flow within the tumor. siRNA which would downregulate eNOS and which could be encapsulated by liposomes and coated onto PC stents could then be delivered to the vasculature of a solid tumour. The efficient local expression of siRNA against the constitutively produced eNOS would decrease local levels of local eNOS expression within the tumour thereby decreasing NO bioavailability and its contribution to tumour angiogenesis. It would also assist it in enhancing vascular dysfunction within the tumour causing a disturbance in the tumour's blood supply and thereby helping its growth to regress.

The gene encapsulated by the liposome may alternatively be selected from the group consisting of:

AKT1 (V-akt murine thymoma viral oncogene homolog 1), ANGPT1 (Angiopoietin 1), ANGPT2 (Angiopoietin 2), ANGPTL3 (Angiopoietin-like 3), ANGPTL4 (Angiopoietin-like 4), ANPEP (Aminopeptidase), BAH (Brain-specific angiogenesis inhibitor 1), CCL2 (Chemokine (C—C motif) ligand 2), CCL11 (Chemokine (C—C motif) ligand 11), CDH5 (Cadherin 5), COL18A1 (Collagen, type XVIII, alpha 1), COL4A3 (Collagen, type IV, alpha 3), CSF3 (Colony stimulating factor 3 (granulocyte)), CXCL1 (Chemokine (C—X—C motif) ligand 1), CXCL2 (Chemokine (C—X—C motif) ligand 2), CXCL3 (Chemokine (C—X—C motif) ligand 3), CXCL5 (Chemokine (C—X—C motif) ligand 5), CXCL6 (Chemokine (C—X—C motif) ligand 6), CXCL9 (Chemokine (C—X—C motif) ligand 9), CXCL10 (Chemokine (C—X—C motif) ligand 10), CXCL11 (Chemokine (C—X—C motif) ligand 11), ECGF1 (Endothelial cell growth factor 1 (platelet-derived)), EDG1 (Endothelial differentiation, sphingolipid G-protein-coupled receptor, 1), EFNA1 (Ephrin-A1), EFNA2 (Ephrin-A2), EFNA3 (Ephrin-A3), EFNA5 (Ephrin-A5), EFNB2 (Ephrin-B2), EGF (Epidermal growth factor (beta-urogastrone)), ENG (Endoglin (Osler-Rendu-Weber syndrome 1)), EPAS1 (Endothelial PAS domain protein 1), EPHB4 (EPH receptor B4), EREG (Epiregulin), F2 (Coagulation factor II (thrombin)), FGF1 (Fibroblast growth factor 1 (acidic)), FGF2 (Fibroblast growth factor 2 (basic)), FGF6 (Fibroblast growth factor 6), FGFR3 (Fibroblast growth factor receptor 3), FIGF (C-fos induced growth factor (vascular endothelial growth factor D)), FLT1 (Fms-related tyrosine kinase 1), HAND2 (Heart and neural crest derivatives expressed 2), HGF (Hepatocyte growth factor (hepapoietin A; scatter factor)), HIF1A (Hypoxia-inducible factor 1, alpha subunit), HPSE (Heparanase), ID1 (Inhibitor of DNA binding 1), ID3 (Inhibitor of DNA binding 3), IFNA1 (Interferon, alpha 1), IFNB1 (Interferon, beta 1, fibroblast), IFNG (Interferon, gamma), IGF1 (Insulin-like growth factor 1 (somatomedin C)), IL10 (Interleukin 10), IL12A (Interleukin 12A), IL18 (Interleukin 18 (interferon-gamma-inducing factor)), IL1B (Interleukin 1, beta), IL6 (Interleukin 6 (interferon, beta 2)), IL8 (Interleukin 8), ITGAV (Integrin, alpha V), ITGB3 (Integrin, beta 3), JAG1 (Jagged 1), KDR (Kinase insert domain receptor (a type III receptor tyrosine kinase)), LAMAS (Laminin, alpha 5), LECT1 (Leukocyte cell derived chemotaxin 1), LEP (Leptin (obesity homolog, mouse)), MDK (Midkine (neurite growth-promoting factor 2)), MMP19 (Matrix metallopeptidase 19), MMP2 (Matrix metallopeptidase 2), MMP9 (Matrix metallopeptidase 9), NOTCH4 (Notch homolog 4 (Drosophila)), NPPB (Natriuretic peptide precursor B), NPR1 (Natriuretic peptide receptor A/guanylate cyclase A), NRP1 (Neuropilin 1), NRP2 (Neuropilin 2,), NUDT6 (Nudix (nucleoside diphosphate linked moiety X)-type motif 6), PDGFA (Platelet-derived growth factor alpha polypeptide), PDGFB (Platelet-derived growth factor beta polypeptide), PECAM1 (Platelet/endothelial cell adhesion molecule (CD31 antigen)), PF4 (Platelet factor 4 (chemokine (C—X—C motif) ligand 4)), PGF (Placental growth factor), PLAU (Plasminogen activator, urokinase), PLG (Plasminogen), PLXDC1 (Plexin domain containing 1), PROK2 (Prokineticin 2), PTEN (Phosphatase and tensin homolog), PTGS1 (Prostaglandin-endoperoxide synthase 1), PTGS2 (Prostaglandin-endoperoxide synthase 2), PTN (Pleiotrophin), ANG (Angiogenin, ribonuclease, RNase A family, 5), OPN/SPP1 (Osteopontin), SERPINF1 (Serpin peptidase inhibitor, Glade F, member 1), SH2D2A (SH2 domain protein 2A), SPHK1 (Sphingosine kinase 1), STAB1 (Stabilin 1), STAB2 (Stabilin 2), SOD (superoxide dismutase), TEK (TEK tyrosine kinase, endothelial), TGFA (Transforming growth factor, alpha), TGFB1 (Transforming growth factor, beta 1), TGFB2 (Transforming growth factor, beta 2), TGFB3 (Transforming growth factor, beta 3), TGFBR1 (Transforming growth factor, beta receptor I), THBS1 (Thrombospondin 1), THB S2 (Thrombospondin 2), TIE1 (Tyrosine kinase with immunoglobulin-like and EGF-like domains 1), TIMP1 (TIMP metallopeptidase inhibitor 1), TIMP2 (TIMP metallopeptidase inhibitor 2), TIMP3 (TIMP metallopeptidase inhibitor 3), TNF (Tumor necrosis factor), TNFAIP2 (Tumor necrosis factor, alpha-induced protein 2), TNFRSF12A (Tumor necrosis factor receptor superfamily, member 12A), TNFSF15 (Tumor necrosis factor (ligand) superfamily, member 15), TNNT1 (Troponin T type 1), VEGF (Vascular endothelial growth factor), VEGFB (Vascular endothelial growth factor B), VEGFC (Vascular endothelial growth factor C), AGGF1 (Angiogenic factor with G patch and FHA domains 1).

Particularly preferred genes are eNOS, iNOS, nNOS, GTPCH-I (GTP cyclohydrolase IANGPT1 (Angiopoietin 1), ANGPT2 (Angiopoietin 2) FGF1 (Fibroblast growth factor 1 (acidic)), FGF2 (Fibroblast growth factor 2 (basic)), FGF6 (Fibroblast growth factor 6), FGFR3 (Fibroblast growth factor receptor 3), HIF1A (Hypoxia-inducible factor 1, alpha subunit),), IGF1 (Insulin-like growth factor 1 (somatomedin C)), IL10 (Interleukin 10), IL12A (Interleukin 12A), MMP2 (Matrix metallopeptidase 2), MMP9 (Matrix metallopeptidase 9), TGFA (Transforming growth factor, alpha), TGFB1 (Transforming growth factor, beta 1), TGFB2 (Transforming growth factor, beta 2), TGFB3 (Transforming growth factor, beta 3), TGFBR1 (Transforming growth factor, beta receptor I), TIMP1 (TIMP metallopeptidase inhibitor 1), TIMP2 (TIMP metallopeptidase inhibitor 2), TIMP3 (TIMP metallopeptidase inhibitor 3), TNF (Tumor necrosis factor), VEGF (Vascular endothelial growth factor), VEGFB (Vascular endothelial growth factor B), VEGFC (Vascular endothelial growth factor C), or siRNAs which could silence these genes.

Suitably the stent may be a phosphatidylcholine-coated or phosphatidylcholine derivative-coated stent or a bare metal stent, which may be either cobalt chromium or stainless steel. The invention also provides a method of producing a nucleic acid-encapsulating liposome or lipoplexcoated stent comprising
(a) mixing a nucleic acid or peptide of interest with a mixture of any cationic lipid or polymer and a “helper lipids” or lipids to form a lipoplex/liposome (Lipoplex 4 Lx4).
(b) coating a stent with the lipoplex

Preferably step (a) of the above method comprises (a) mixing a nucleic acid or peptide of interest with a mixture of DDAB and DOPE to form a lipoplex (Lx1); or,

(b) mixing a nucleic acid or peptide of interest with a mixture of DDAB and CHOL/POPC to form a lipoplex (Lx2); or,
(c) mixing a nucleic acid or peptide of interest with a mixture of DOTMA and DOPE to form a lipoplex (Lx3).

The amount of cationic lipids and the amount of helper lipids may vary from a 1:1 to a 1:5 molar ratio. The ratio of cationic lipid amount (nmole) to plasmid amount (m) may vary from approximately 3:1 to 6:1 in these formulations. Preferably, aqueous components or organic solvents are removed from the lipoplex-stents by lyophilisation in a vacuum.

The invention further provides a method of delivering a nucleic acid to a site in the body comprising the use of an implantable device as described above.

In a preferred embodiment the invention further provides a method of delivering a nucleic acid to a site in a blood vessel comprising use of a stent as described above. The blood vessel may be in the heart, the brain, the kidney or other organ, or it may be in a tumour, which is fed by an artery. Thus the stent may find use in the prevention or treatment of heart attacks, stroke, peripheral artery disease or cancer.

The invention also provides a method of prevention stenosis or restenosis comprising use of a liposome-coated stent as described above.

The term “liposome” as used herein means any vesicle consisting of an aqueous core enclosed by at least one lipid layer.

The term “lipoplex” as used herein means any lipsome-nucleic acid complex.

The term “implantable device” as used herein means any device, which is intended to be totally or partially introduced surgically or medically into the human body and which is intended to remain after the procedure. The device can be temporary or permanent.

The term ‘nucleic acid’ as used herein includes genes, oligonucleotides, peptides and siRNAs, miRNAs or shRNAs which can silence specific genes.

Unless otherwise defined, all terms of scientific terminology used herein are intended to have the meanings commonly understood by those of skill in the art. Some terms are defined herein for clarity and the inclusion of such definitions should not be construed to represent a substantial difference over what is generally understood in the art or intended to limit the scope of the invention in any way.

The current invention will now be described with reference to the following examples and figures. It is to be understood that the following detailed description and accompanying figures, are exemplary and explanatory only and are intended to provide a further explanation of the present invention, as claimed and not to limit the scope of the invention in any way.

### BRIEF DESCRIPTION OF THE DRAWINGS

FIG. 1: Scanning electron microscopy demonstrating efficient coating using all liposomal formulations.

FIG. 2: Graph demonstrating the relative expression of the LacZ reporter gene in the media and neointima of rabbit external iliac arteries at 28 and 42 days post-deployment of both PC coated stainless steel and cobalt chromium stents coated with lipsomal formulations. Values for medial and neointimal expression were calculated as a percentage of total transgene expression observed for the entire vessel (n=3-7).

FIG. 3: The expression of beta-galactosidase reporter gene using each of the three liposomal delivery formulations in vessels at day 28 as evidenced by blue stain (darkly staining on grayscale), using X-gal staining detected both macro and microscopically.

FIG. 4: The expression of LacZ in cells as evidenced by blue stain using X-Gal staining and highlighted using arrows. Expression is noted in the neointima near an indentation from a stent strut (S). Expression is also noted between stent struts.

FIG. 5: A histological section of liposomal-transduced neointima demonstrating co-localisation of beta-galactosidase expression using X-Gal stain and macrophage phenotype using fluorescent detection of ram-11.

FIG. 6: Representative histological sections of organs distal to the site of stent deployment stained using XGa1 solution demonstrating no positive stain for β-galactosidase activity. (A) Liver (B) Lung and (C) Spleen. Magnification 20×.

FIG. 7. De-endothelialisation of vessels treated with PC stents with and without Liposomal eNOS (lipoplex 1). Areas of gray represent de-endothelialised areas. This was also determined using histology and is illustrated in the graph.

FIG. 8: A comparison of reporter gene delivery using adenovirus (Ad), adeno-associated virus (AAV) and liposomal delivery (lipo) delivery over a time course of 1 month

FIG. 9: Lipoplex 1 Beta Gal gene delivery at 28 days using off balloon delivery [aorta and common iliacs dissected intact]

FIG. 10. Low cytotoxicity of liposomal formulations delivered in vitro to both vero cells (kidney epithelial cells extracted from an African green monkey, Cercopithecus aethiops) and coronary artery smooth muscle cells. Before and after freezing at −80° C.

FIG. 11. (A) Expression of a therapeutic gene in a blood vessel wall following delivery via a liposome. (B) The reduction of total occlusions in arteries treated in vivo using liposomally-delivered eNOS (lipoeNOS). Not powered sufficiently for statistical significance.

FIG. 12. Delivery and expression of transgenes in soft tissue from a stainless steel coupon surface.

### DETAILED DESCRIPTION OF THE DRAWINGS

### Materials and Methods

Construction of Lipoplex Complexes with Plasmid LacZ

A plasmid DNA encoding LacZ gene driven by the CMV promoter was constructed according to Qiagen Endofree Plasmid Giga Kit manufacturer's instructions. For these experiments we used either control “off the shelf” or ready-to-use liposomes (lipofectin, Invitrogen), or self prepared liposomes composed of DDAB/DOPE or DDAB/Chol/POPC to bind β-galactosidase or eNOS plasmid DNA to form lipid-DNA complexes (so-called lipoplexes). Lipofectin (Lx 3) is a 1:1 molar mixture of the DOTMA (N-[1-(2,3-dioleyloxy)propyl]-n,n,n-trimethylammonium chloride and DOPE (dioleoylphos-photidylethanolamine). DOTMA or DDAB are cationic lipids which help in binding the negatively charged nucleic acids and DOPE is a so called ‘Helper-lipid’ which allows the entrapped nucleic acid to escape the endosomes after cell entry by endocytosis. Cholesterol aids in forming stable, gel-phase (“waxy”) liposomes. POPC is a derivative of naturally occurring phospholipids (phosphatidylcholines) that form the cell membranes of eukaryotic cells.

All lipid components used in the self-made liposomal formulations were obtained from the vendor (Avanti Lipids, USA) as organic solvents and mixed in glass tubes at the ratios described above (see page 7). All organic solvents were subsequently removed in a rotary evaporator at 100-200 mbar, >65° C. for 45 min to achieve lyophilisation of the lipids. In a typical experiment, a plasmid solution containing 100 μg of DNA (reporter gene or eNOS) was added to the lipid film in the glass tube. Lipoplexes 1 & 2 were formed by brief sonication in a water bath. Lipofectin-plasmid complexes: For these experiments 10 μl of a 10 mg/ml LacZ plasmid solution (100 μg plasmid DNA encoding LacZ) was diluted to a total volume of 100 μl with endotoxin free TE buffer (Invitrogen). 200 μl of a 1 mg/ml lipofectin/liposome solution was added to the plasmid solution and mixed several times by inverting the tube. The components were allowed to form lipoplexes for 45 minutes at room temperature and stored at 4 degrees centigrade.

### Animals

A preclinical animal model was developed in to assess the efficiency of liposome coating of stents. Male New Zealand White rabbits (Harlan Ltd UK) weighing 2.5 to 3.5 Kg were used. Animals were individually housed with a 12 hour light-dark cycle and fed a standard chow diet and given water ad libitum. All animals received low dose aspirin for seven days prior to intervention and thereafter until euthanasia Animals were sacrificed at 28 days with a high dose of phenobarbitone which was administered intravenously following sedation.

### Preparation of Liposome-Coated Stents

Commercially available phosphorylcholine or phosphorylcholine derivatives (Avanti Polar Lipids, AL/USA) are solubilised in sterile PBS or Tris/EDTA solution at room temperature. Precipitates can be dissolved quickly by incubating the samples in a sonicating water bath and moderate heating. The stent is inserted into the programmable stent movement and rotation device on the coating system. Alternatively, the bare metal stent (e.g steel, nitinol or cobalt alloy stents) can be coated directly.

Lipoplexes composed of DOTMA/DOPE, DDAB/DOPE or DDAB/Chol/POPC and genes (eNOS etc.,) are formulated as described earlier for LacZ.

All aqueous components are removed from PC coated stents or lipo-stents by lyophilisation in a vacuum with liquid nitrogen cooling.

Stent platforms included biodivYsio HI matrix PC coated premounted stents, cobalt chromium stents (3.0×15 mm) and partial nitinol stents were used for these experiments. The majority of stents were manually coated using a micro pipette under sterile conditions with a 300 pt bolus of cationic liposome carrying 100 μg plasmid DNA encoding LacZ and air dried for 45 minutes prior to stent deployment.

### In Vivo Catheter Procedures

All procedures were performed under fluoroscopic guidance. Prior to the procedure all animals were given an appropriate dose of heparin intravenously Animals were anaesthetized with isoflurane after sedation with ketamine (35 mg/kg), xylazine (5 mg/kg) and acepromazine (1 mg/kg) under sterile conditions. The right carotid artery was surgically exposed by blunt dissection and a 5 Fr introducer sheath (Radifocus, Terumo) was introduced into the artery and advanced to the lower abdominal aorta. All wires and catheters were passed through this sheath. A balloon injury was performed with a 2.5×14 mm commercially available balloon which was placed in the right external iliac artery. A total of three balloon injuries were performed of 1 minute duration each (6 ATM for 60 second). A one minute interval of deflation was allowed between balloon inflations. After balloon injury a 3.0×11 mm BiodivYsio HI matrix coated stent was deployed at the injury site (6 ATM for 30 second). This was repeated for the left iliac artery. Post-stent deployment angiography was carried out in all animals to exclude any acute thrombus formation at the site of stent deployment.

### Histochemical Analysis of Gene Expression

β-galactosidase activity was studied in a total of eight animals. Following sacrifice, stented arteries were exposed, retrieved and cut longitudinally with the stent removed prior to staining of arteries. A significant neointimal formation inside the luminal face of the stent was noted at day 28 which was stripped from the luminal face of the stent and stained separately for β-galactosidase activity. All stented arteries were fixed with 4% paraformaldehyde for 30 minutes at 4° C. and then rinsed twice with Phosphate Buffer Saline (PBS). Arteries were then stained in a solution of 500 μg/ml 5-bromo-4-chloro-3-indolyl-β-D-galactopyranoside (X-Gal; Boehringer-Mannheim Biochemicals, Mannheim, Germany) overnight at 37° C. Following staining, the arteries were frozen in optimum cutting temperature compound (OCT). Sections (5 μm) were then cut, placed on slides and stained with either eosin alone or with hematoxylin and eosin. Expression was considered positive for blue cells visible under light microscopy. In addition to staining of the stented blood vessels, multiple distal organs were also stained with X-Gal the presence or absence of (3-galactosidase protein.

### Identification of Transgene Expressing Cells

Histological sections, which had previously been positively stained for transgene expression, as described above, were subsequently analysed for smooth muscle phenotype. Following Xgal staining, slides were washed twice in PBS containing 1% tween. Slides were incubated in blocking solution (5% goat serum in PBS with 0.5% Triton X-100) for 30 minutes. Slides were then incubated with primary α-SMA antibodies (Mouse monoclonal (1 A4) anti-alpha smooth muscle actin) (Abcam) diluted in 1% goat serum and 0.5% bovine serum albumin and 0.5% Triton X-100 in PBS overnight. Slides were washed in PBS/1% tween and incubated with anti-mouse IgG conjugated with Alexa Flour 488 (Molecular Probes) diluted in 1% goat serum and 0.5% BSA in PBS for 2 hours minimum in the dark. Slides were washed in PBS and then mounted using Vectashield containing DAPI. Putative co-localisation of transgene expression and smooth muscle phenotype was assessed visually.

### Image Analysis of Transduced Vessels

The luminal surface of all the stained arteries was photographed en face through a dissecting microscope. Surface areas were quantified by two operators blinded to the treatment. Quantification of positively (blue) stained tissue was done using Java Image processing programme software (Image J) from the National Institute of Health. At days 3 and 7, neointimal formation could not be visualized or separated from the media allowing only the vessel wall to be stained and quantified. However, at day 28 there was a significant neointimal formation observed which could be removed, stained and imaged separately to the vessel media. For quantification purposes the total amount of expression observed and quantified in both the medial and neointimal layers at day 14 and later was used to determine the total percentage of expression for each timepoint.

### Morphometric Analysis

The effects of Liposome encapsulating (3-galactosidase (Lipoβgal) coated stents on the hismorphometry of the blood vessels was compared with AdiβGal controls in hypercholesterolemic rabbits (n=4 each group). Note: LipoBgal refers to lipoplex 1. After anaesthesia the animals were perfusion fixed and the stented blood vessels were retrieved. The stented vessel segments were embedded in methylmethacrylate plastic. After polymerization, two to three millimeter sections were sawed from the proximal, mid and distal portions of each single stent. Sections from the stents were cut on a rotary microtome at four and five microns mounted and stained with hematoxylin, eosin and elastic Van Gieson stains. All sections were examined by light microscopy for the presence of inflammation, thrombus and neointimal formation and vessel wall injury.

A vessel injury score was calculated according to the Schwartz method (Schwartz et al. 1992). The cross sectional areas (EEL, IEL, and Lumen) were measured with digital morphometry. Neointimal thickness was measured as the distance from the inner surface of each stent strut to the luminal border.

### Assessment of In-Stent Restenosis by Angiogram

Follow-up angiograms were performed via left carotid artery, at 28 days post initial intervention in LipoβGal stented hypercholesterolemic animals (n=4). All the angiograms were analyzed for quantitative assessment of in-stent restenosis using standard quantitative coronary angiogram (QCA) software by two operators blinded to the treatment groups.

### Statistical Analysis

Data for all experiments were expressed and graphed either as the median value or as a mean+/−standard error of mean. Statistical analysis was performed by two way repeated measure ANOVA with a Schefee's test to detect differences in multiple comparisons and by an unpaired t-test for comparisons between the two groups. A P<0.05 was considered significant.

Detection of eNOS with Reverse Transcriptase PCR

RNA was extracted from the rabbit arteries 21 days after exposure to Lipo-eNOS using the RNeasy kit (Qiagen). Extracted and purified RNA (200 ng) was then subjected to reverse transcription PCR using the Quantitect SYBR Green RT-PCR kit (Qiagen) in conjunction with the ABI 7000 Sequence detection system (ABI). (50° C., 30 min; 94° C., 15 min; 35 cycles of 94° C., 15 sec; 60° C. 1 min). PCR products were visualized on agarose gels. PCR primers were designed to amplify the human eNOS transgene without amplifying endogenous rabbit orthologues. eNOS primers were forward 5′-GGAGATACGAGGAGTGGAAG-3′ and reverse 5′-GCCAAACACCAGGGTCATAG-3′ with an expected product size of 449 base pairs. Primers against the rabbit house keeping gene hypoxanthine phosphoribosyltransferase (HPRT) were used as a control with expected product size of 135 base pairs.

### Assessment of Endothelialisation

Endothelial levels in the vessels were examined using a 1% Evans blue solution which was injected intravenously 30 minutes prior to sacrifice. Vessels were retrieved and examined macroscopically after sacrifice. Areas with blue stain indicate lack of an endothelial layer. Areas of white tissue illustrate intact endothelium. Alternatively, endothelial numbers were estimated histologically by an independent, blinded histopathologist.

### Assessment of Capacity to Deliver to Soft Tissue.

Experiments were carried out as described above. In addition, stainless steel coupons (5 mm in diameter) coated with the lipoplex formulations as per the stents were implanted in perivascular soft tissue and harvested at 28 days post-deployment.

### Results

### PC Coated Stent-Mediated Delivery of LacZ to the Blood Vessel Wall Using Lipoplexes 1, 2 and 3

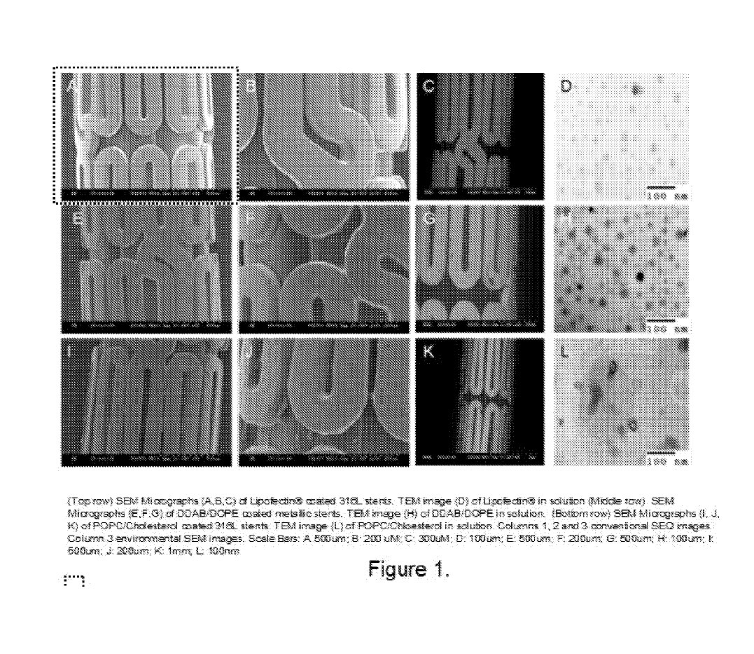

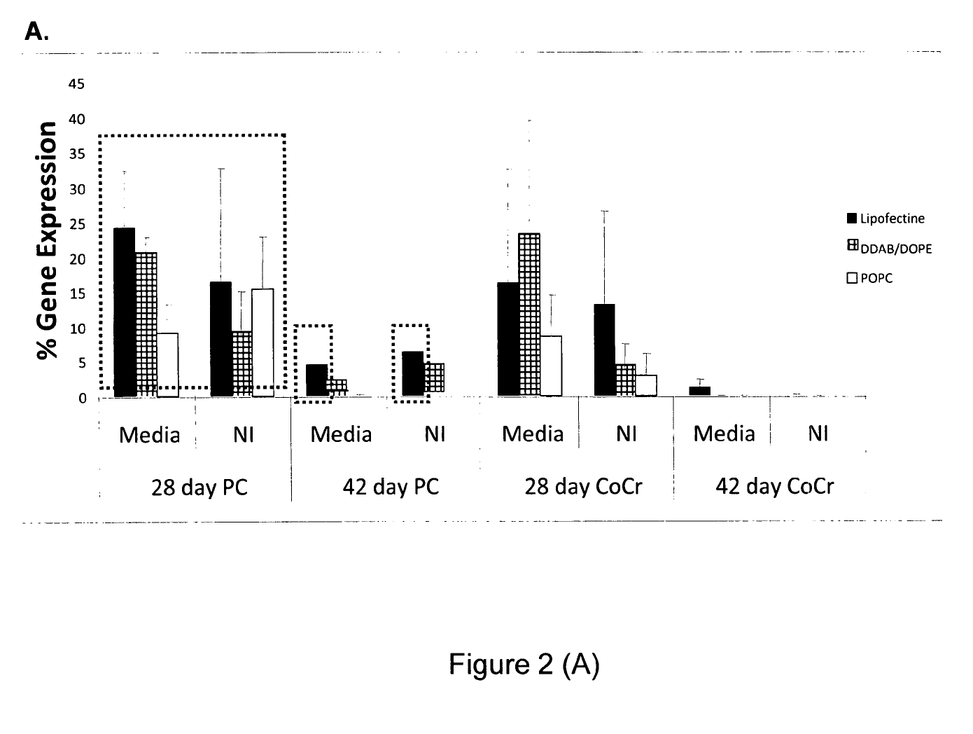

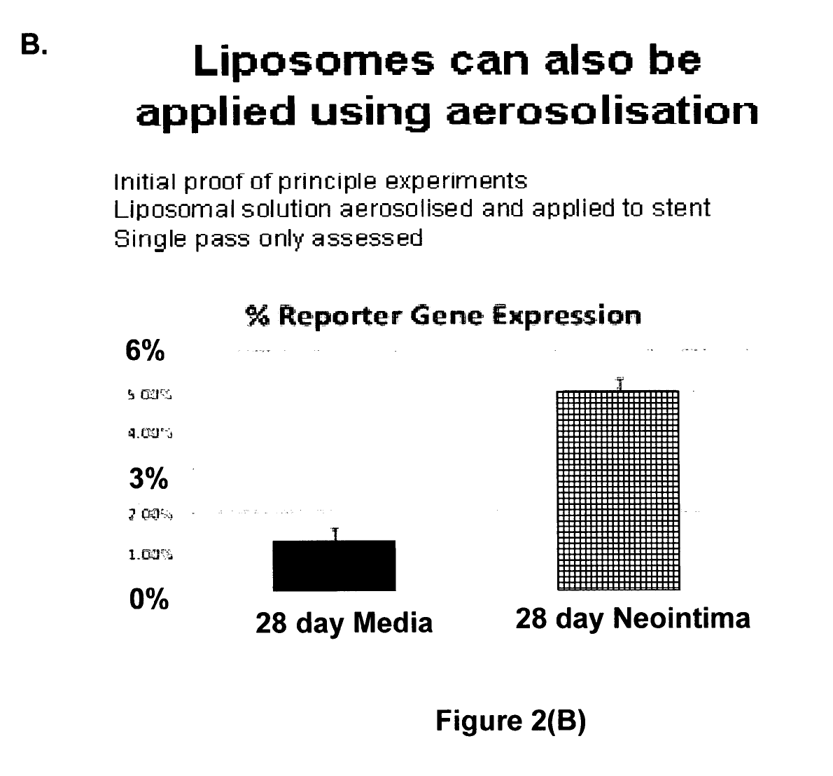

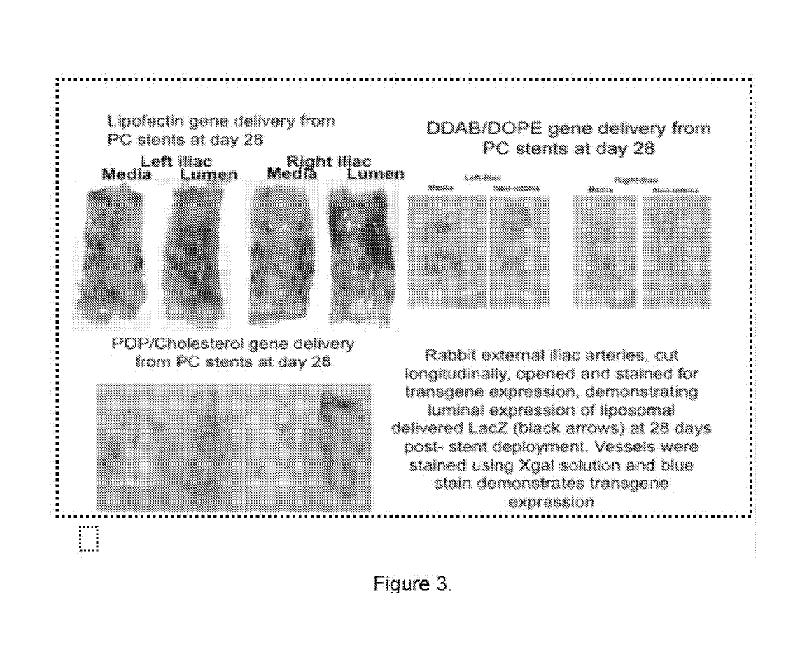

Scanning electron microscopy of FIG. 1 illustrates a very smooth and dense coating of the lipoplexes on the stent indicative of very stable stent-lipid-plasmid interactions. Expression of the reporter gene in LipoβGal transduced arteries was assessed at days 3, 7, 28 and 42 days. The level of gene expression achieved after liposome mediated gene delivery to the blood vessel wall was low at earlier time points. At day 3, median level of gene expression achieved was 1.47% (range 0.8%-1.85% n=4). At day 7, the level of reporter gene expression was persistently low with a median value of 1.2% (range 1.1%-1.25% n=4). The increase in β-galactosidase activity detected for stent-delivered LipoβGal corresponded with the formation and development of neointimal tissue. Neointimal formation was macroscopically observed as tissue formation occurring between the struts of the stent and separated from the media by the stent. This neointimal tissue could easily be stripped away from the medial wall. This allowed expression in media and neointima to be separately analyzed. The relative contributions of medial and neointimal β-galactosidase activity for day 28 can be seen in Table 1, FIG. 2 and FIG. 3. The level and site of expression of β-galactosidase activity can be modulated depending on the liposomal formulation. Lipofectin and DDAB/POPC/Cholesterol are most effective delivering genes to the neointima whereas DDAB/DOPE is more effective delivering the genes to the media. Lipsomal formulations may also be effectively delivered using an aerosol delivery system. β-galactosidase activity was still observed in the medial and neointimal tissue at day 42 post liposome-eluting stent deployment.

TABLE 1
Data demonstrating expression at different sites and time
points using various liposomal formulations from PC stents

| FORMULATION | SITE | TIMEPOINT | % EXPRESSION |
|---|---|---|---|
| Lipofectin | Media | 28 days | 24.26 |
| Lipofectin | Neointima | 28 days | 16.45 |
| Lipofectin | Media | 42 days | 4.5 |
| Lipofectin | Neointima | 42 days | 6.4 |
| Lipofectin | Media | 28 days | 0.99 |
| | | (Aerosol coating) | |
| Lipofectin | Neointima | 28 days | 5.45 |
| | | (Aerosol coating) | |
| DDAB/DOPE | Media | 28 days | 21.02 |
| DDAB/DOPE | Neointima | 28 days | 8.11 |
| DDAB/DOPE | Media | 42 days | 1.38 |
| DDAB/DOPE | Neointima | 42 days | 4 |
| POPC/Cholesterol | Media | 28 days | 9.1 |
| POPC/Cholesterol | Neointima | 28 days | 15.3 |
| POPC/Cholesterol | Media | 42 days | 0.07 |
| POPC/Cholesterol | Neointima | 42 days | 0 |

### Cellular Distribution of Transgene Expression

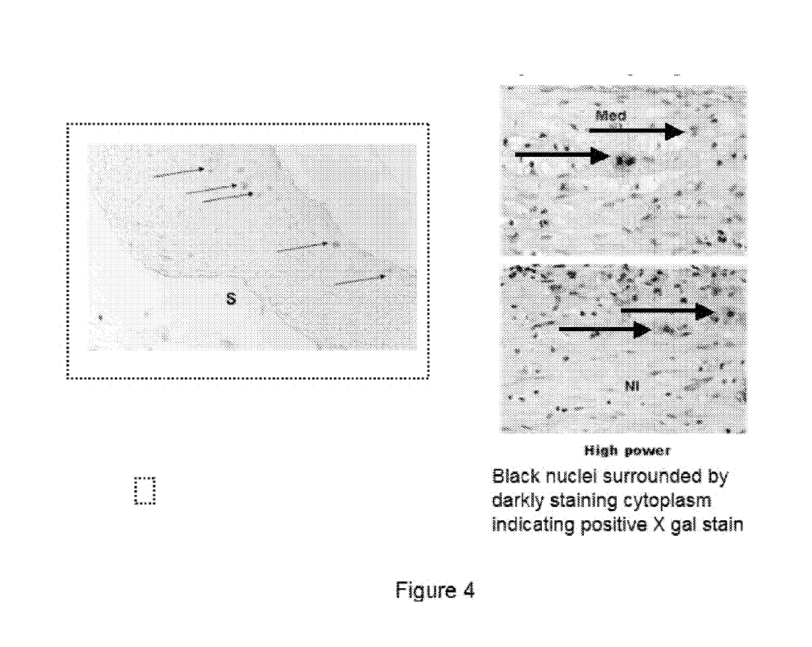

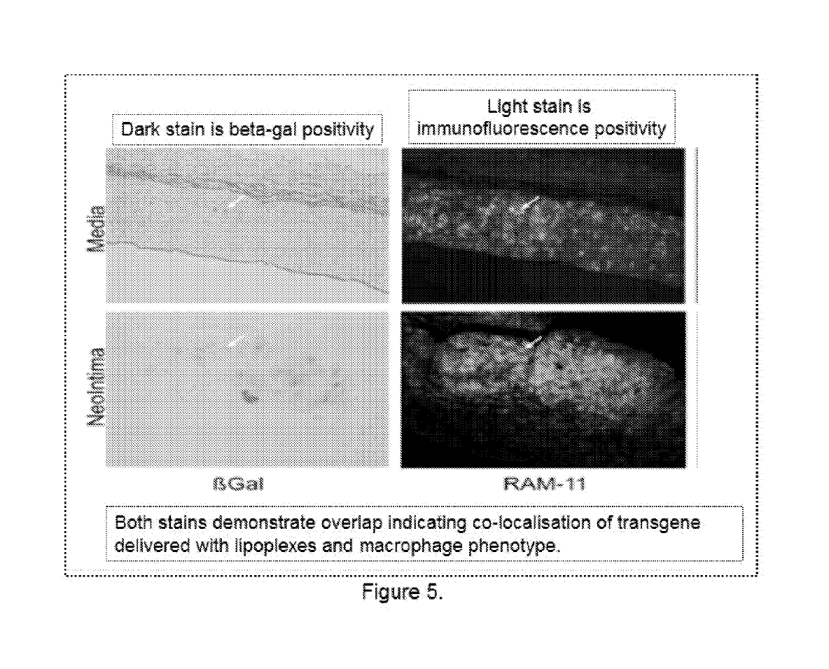

Histological examination of liposome β-galactosidase transduced vessels was carried out following removal of the stent from the vessel. Both media and neointima at day 28 were examined to assess the cellular distribution of transgene expression. Delivery of LipoβGal resulted in transgene expression adjacent to the stent strut excavations in the media and neointima (FIG. 4). Furthermore, sections which had cells that stained positively for β-galactosidase activity also demonstrated staining for macrophages (FIG. 5). Based on this co-localisation pattern transgene expression obtained with this vector was distributed in cells with a phenotype consistent with macrophages. Expression has also been noted between stent struts as well as adjacent to them (FIGS. 3 & 4).

### Detection of Distal Transgene Expression

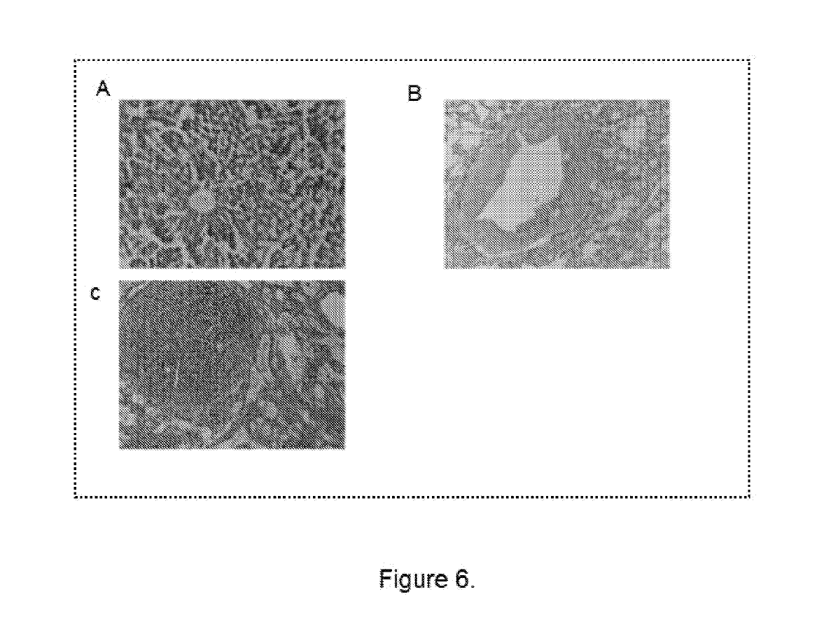

β-galactosidase expression could not be detected in distal organs (brain, heart, lung, liver, spleen, pancreas, kidney and testes) using the same staining procedures as that applied to the stented arteries. This result suggests that it is possible to obtain localized transgene expression in the blood vessel wall using stent based gene delivery in the absence of distal spread (FIG. 6).

### Evans Blue Detection of De-Endothelialisation

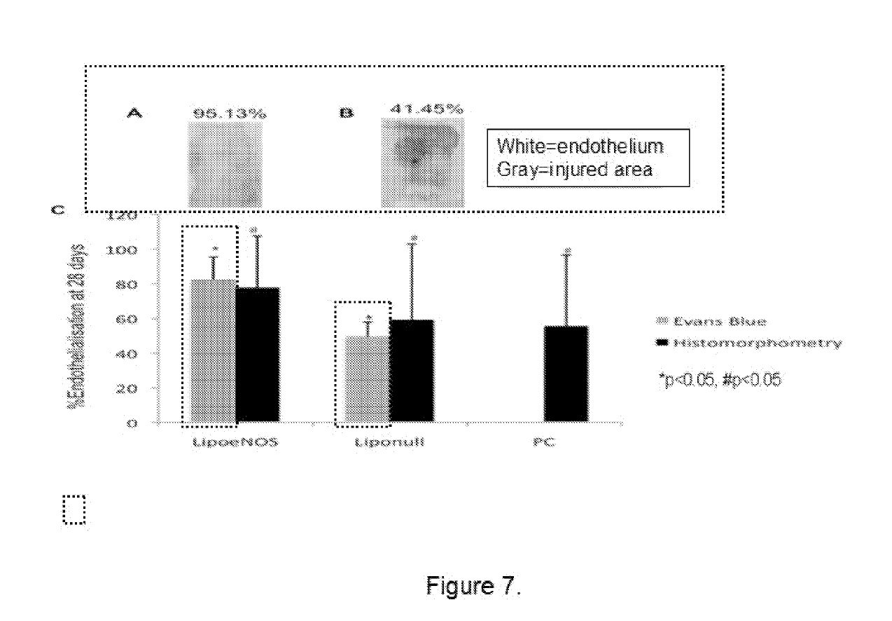

Following delivery of a therapeutic gene using a liposomal delivery system from PC coated stents we were able to assess its effect on de-endothelialisation post-balloon injury using Evans blue which stains de-endothelialised areas blue. Areas of endothelialisation were significantly enhanced (P=0.00497, t-test) when eNOS was delivered from a liposomal delivery system (lipoplex 1) from PC stents compared to control stents with PC (82.5+/±13 versus 49.6+/−8, FIG. 7). This was also confirmed histologically by independent and blinded operators (FIG. 7).

### Quantitative Angiography

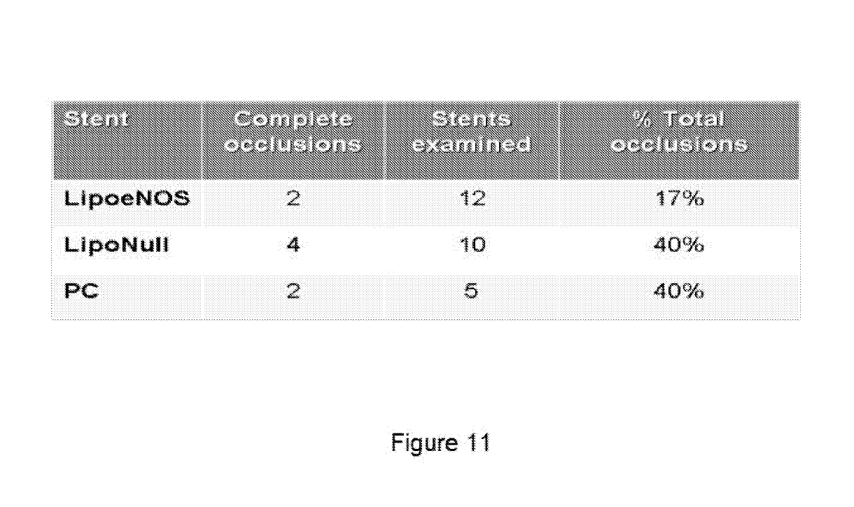

Follow-up iliac artery angiogram was performed at five weeks post balloon injury and stenting in the LipoβGal treated hypercholesterolemic animals. Liposomal delivery of eNOS from PC stents versus the use of normal PC stents resulted in less complete occlusions in lipoeNOS-treated arteries. Histological analysis also confirmed this reduction (FIG. 11).

Comparison with Other Vectors

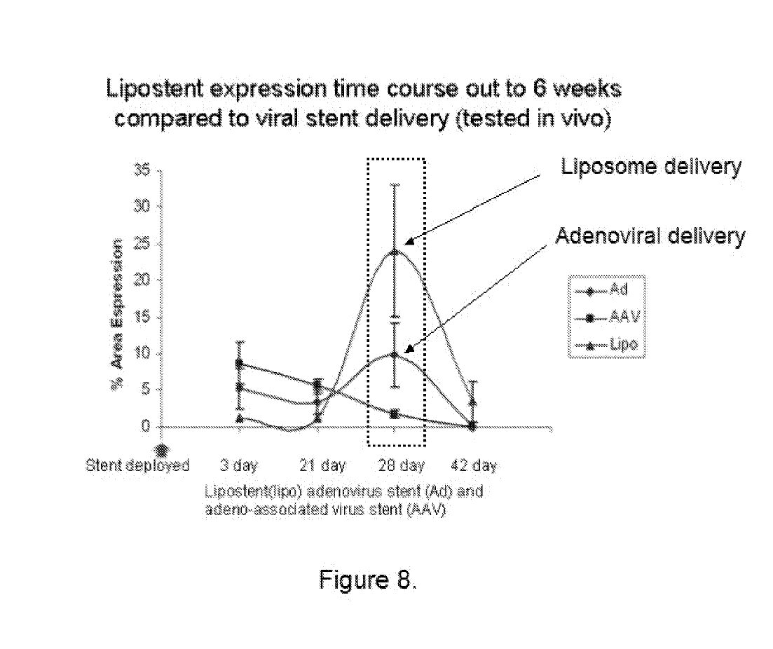

The long-term efficiency of reporter gene delivery using lipoplex 1 (Lipo) was improved compared with adenovirus (Ad) and adeno-associated virus (AAV) (FIG. 8).

Delivery from Alternative Surfaces

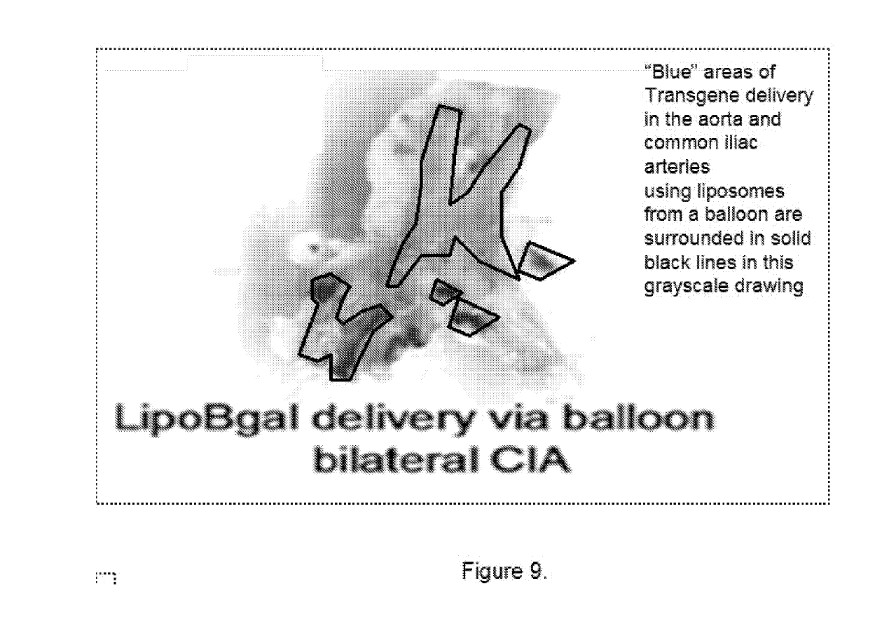

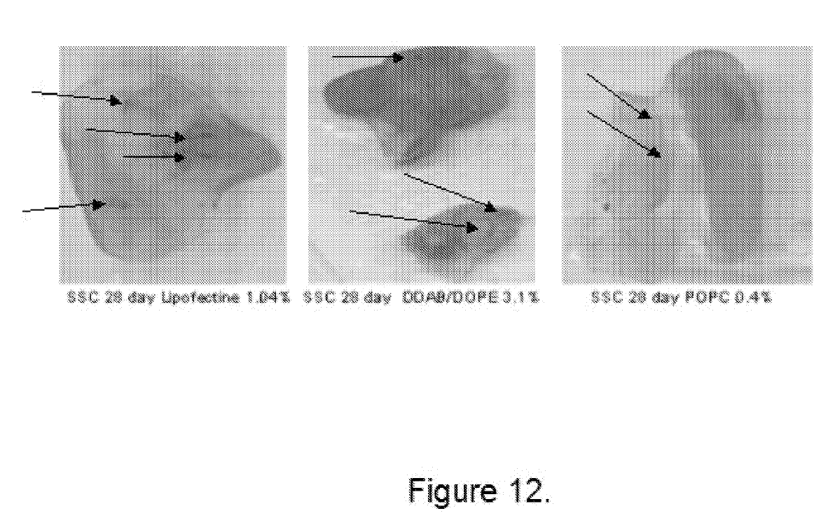

The current inventors also analysed the efficiency of delivery of lipoplexes from bare metal stents and have successfully demonstrated proof of principle for delivery of lipoplexes, in this case lipoplex 1, from bare metal stents and balloons as illustrated in FIGS. 2 and 9. Moreover, gene delivery could also be observed when lipoplex 1 was coated onto a balloon surface (FIG. 9). However, the method is not as efficient as stent delivery with comparatively decreased levels of gene expression using this method in the bilaterally treated common iliac arteries. A PC stent with lipoplex 1 delivering beta galactosidase acted as a positive control. We have also demonstrated a proof of principle expression of our delivery system from nitinol stents and from stainless steel coupons (FIG. 12).

### Cytoxicity of Lipoplexes

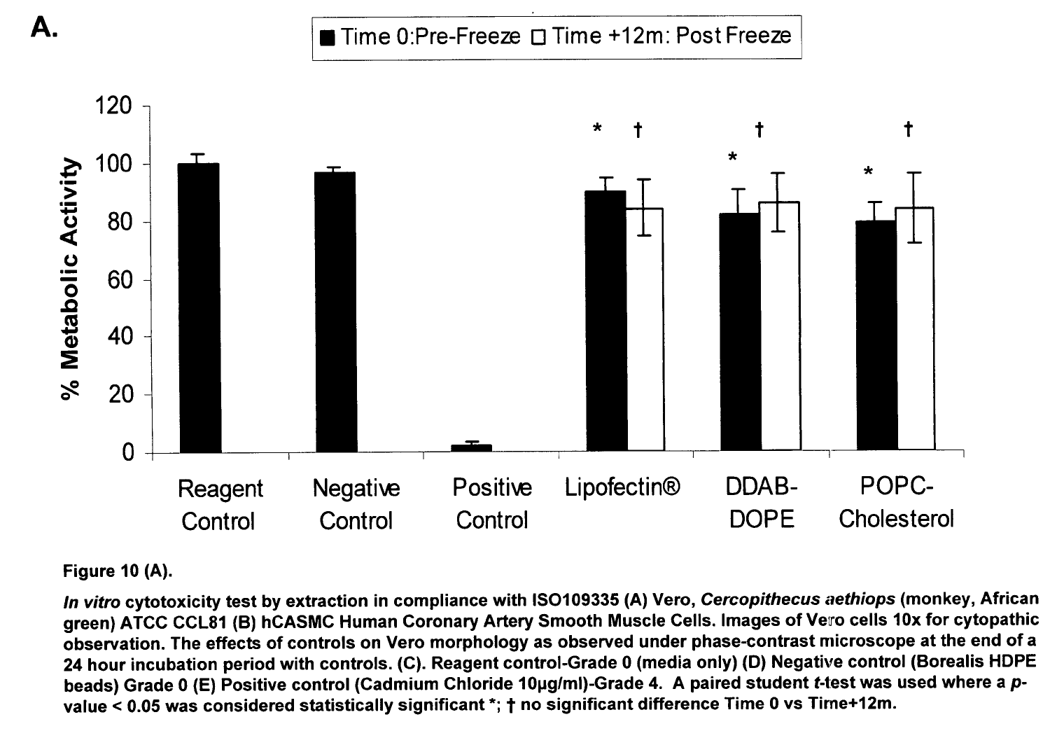

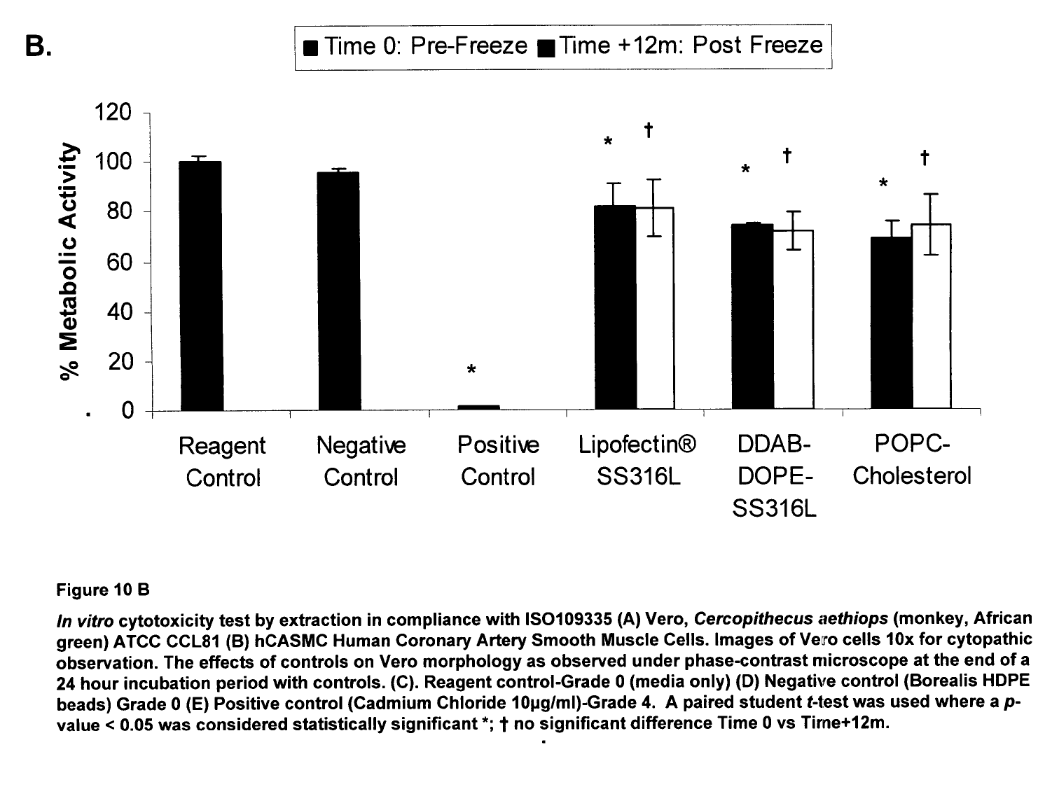

We were able to demonstrate low levels of cytotoxicity of the lipoplexes from stainless steel coupons when examined against vero cells and only slightly higher cytotoxicity when examined against human coronary artery smooth muscle cells in vitro (FIG. 10.

### Discussion

The current inventors have surprisingly illustrated that the lipoplexes of the current invention can be used for stable and effective gene delivery from an implantable device such as a stent. To date successful transfection with liposome mediated delivery has been low, has been beset with problems involving the induction of an inflammatory response in the human body and a lack of tissue trophism. The current invention circumvents the problems of the prior art by using an implantable device based platform, such as a stent, to deliver the plasmid-encapsulating liposomes or lipoplexes, directly to cells.

The current inventors have illustrated stable and effective gene delivery to a blood vessel wall from a PC-coated stent. Liposome delivery resulted in stable and prolonged transgene expression in the blood vessel wall, which was predominantly in the neointimal tissue at the later time point. The transgene expression was localized around the stent struts with no distal dissemination of the vector. Thus liposome mediated local gene delivery can result in a prolonged transgene expression in the blood vessel wall. It is interesting to note that gene expression peaked at 4 weeks post stent placement with relatively low level of expression observed for up to 7 days post intervention. Previously no such long-term efficiency of liposomal-delivery of reporter genes has been shown and it was apparent that these vectors may be too inefficient and short term to be useful from the relatively low-dose stent-based platform in contrast to a high dose catheter-based system. Such a catheter system may however be used to coat the stent at deployment.

The current inventors have importantly shown delivery of the lipoplexes of the current invention from bare metal stents and balloons using a PC coated stent as a control. As can be seen in FIG. 2 delivery of lipoplex 1 is as efficient from bare metal cobalt chromium stents as it is from PC coated stents with distribution equally in the media and neointima. Moreover, it is also possible to deliver from a balloon. However, expression is relatively less. It should be noted that in FIG. 9 the aorta and both common iliac arteries were dissected intact. Of note there is almost no expression of reporter gene in the left common iliac artery despite both arteries being treated in the identical manner and expression in the right common iliac artery is qualitatively not as high as with stents FIG. 3. Also, some expression can be seen in the aorta, most likely due to excess residue on the balloon as it passed through the aorta. The fact that lipoplexes may be delivered from a bare metal, nitinol stents or stainless steel coupons implies that other biomaterials may be suitable for use as a platform for gene delivery in order to modify the host response to a foreign body/implant. Liposome mediated gene delivery from catheters seem to be very efficient. Lipoplex delivery of iNOS under the control of the cytomegalovirus promoter using an infiltrator drug delivery balloon system reduced the neointima thickness in proximal anastomoses at the prosthetic wall, suture region and arterial wall by 43%, 52% and 81%, respectively. In distal anastomoses, the average reduction was 40%, 47%, and 52% respectively (Pfeiffer et al., 2006). Other catheter based approaches resulted in high transfection efficiency of the vessel area as well (Muhs et al., 2003). Although gene expression has been seen in both medial and neointimal cells with this vector, it is important to note that with increasing time, expression of transgene was predominantly observed in the neointimal cells. This implies that the liposome was released slowly from the polymer over a prolonged period and was incorporated into proliferating neointima cells. Therefore, this method may be ideal for delivering genes to the pathological site.

Non-viral liposome mediated delivery systems have correlated poorly with the viral delivery systems for vascular gene delivery in the past. The level and duration of transgene expression achieved with these vectors is very inefficient. However despite this drawback, liposome-mediated gene delivery systems have the potential to deliver therapeutic genes for most clinical applications of gene therapy. We have previously reported that viral vectors especially adenovirus and AAV serotype 2 can be delivered locally to the blood vessel wall over a prolonged duration using PC-stent technology (Sharif et al., 2006). The data from our present study on stent-based liposome mediated gene delivery when compared with our previously published study on viral vector based delivery systems, demonstrates that this vector was also stable and can transduce vascular cells with resultant transgene expression for 28 days. Like adenovirus and AAV2 viral vectors, transgene expression was observed in the media initially but localised to neointima at 28 days. Moreover, the transgene expression was localized to the stented vessel with no distal spread and in fact followed the pattern of stent struts. In addition, interestingly, the temporal profile of transgene expression obtained with liposome had a similar pattern to adenovirus mediated gene delivery except for a non-significantly higher expression at day 28 (liposome median 16.4% versus adenovirus median 7.3% p=0.22). However, the gene expression observed with this vector was significantly higher than AAV2 mediated gene expression at day 28 (liposome median 16.4% versus AAV2 median 2.1% p<0.05) as shown in FIG. 8.

Previous studies have shown that in vitro liposome mediated gene delivery does not correlate with in vivo efficacy of liposome complexes when studied in the lung (Lee E R et al., 1996). In addition there are reports in the literature suggesting that optimal in vivo gene/drug delivery with liposome vectors can be achieved systemically when the molecular ratio of cationic liposome to nucleic acid in the lipoplex mixture (positive/negative charge ratio) is closer to 1 or greater (Liu F et al., 1997; Yang J P and Huang L., 1997). This higher charge of the lipoplex complex also helps in reducing inflammatory and immune response by inactivating serum (Yang JP and Huang L 1997). These previous studies have dealt with gene expression/drug release following systemic delivery of liposomes and does not relate to stent release of liposomes. However, their findings do allow us to speculate that the prolonged and efficient gene expression seen in our study is possibly due to the intrinsic ability of the liposomes to bind efficiently with the PC polymer with stable release over time in vivo and due to their mechanical delivery to the blood vessel wall. Scanning electron microscopy imaging demonstrated that liposomal application to the Hi Matrix PC stent resulted in complete and smooth coating of the stent in comparison with the glycerol application (for adenovirus and AAV2) to the PC stent. Expression of the liposome-mediated reporter gene was noted in arteries 28 days following deployment of stents. Expression of the transgene in the wall of the vessel was estimated at a median value of 16.4% at 28 days. This median value is higher than expression for LacZ delivered using an adenovirus or adeno-associated virus examined in a previous study (Sharif et al., 2006) at 7.3 and 2.1 respectively (FIG. 8) although this was only significantly different for the latter (p<0.05). Examples of the levels of (3-galactosidase activity for stent-delivered LacZ at 28 days are shown in FIG. 2.

The expression of β-galactosidase activity detected for stent-delivered liposome-mediated LacZ corresponded to expression along and between the pattern of the stent struts in the media and neointima. Neointima formation was macroscopically observed as tissue formation occurring between the struts of the stent and separated from the media by the stent. This neointimal tissue could easily be stripped away from the medial wall. This allowed expression in media and neointima to be separately analyzed. The formation of neointima in this model is consistent from day 14 onwards as we have shown in a previous study (Sharif et al., 2006). This invention as demonstrated in the preclinical model has a higher efficiency and better ability to deliver transgenes to the blood vessel wall than the strategies outlined above. Moreover the use of viral-based delivery systems has added biosafety concerns, which this invention does not have. The lack of viral proteins and infective process implies that there will be a lower immunological and inflammatory response than for viral-based vectors from a stent. The present invention has a high efficiency of delivery, which is targeted to particular cell populations that are important in establishing in-stent restenosis in particular macrophages in the neointima in the vessel wall. Therefore, this method is ideal for delivering genes to the pathological site of an atherosclerotic lesion either in the coronary or peripheral vasculature.

At present the invention has shown efficient delivery of reporter genes to the local vessel wall at 1 month post-stenting with no evidence of distal spread thus proving that a non-viral vector can be more efficient than a viral vector for delivering genes to the vasculature. When used in conjunction with the eNOS gene we have shown the ability of our invention to enhance re-endothelialisation of the blood vessel wall.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

### REFERENCES

Balbi M, Fedele M, Bezante G P, Brunelli C, Barsotti A. Acute myocardial infarction related to very late sirolimus-eluting stent thrombosis 6 months after discontinuation of dual antiplatelet therapy. J Cardiovasc Med (Hagerstown). 2010 Mar. 4. [Epub ahead of print]
Cooney R, et al. Effect of gene delivery of NOS isoforms on intimal hyperplasia and endothelial regeneration after balloon injury. Gene Ther. 2007 March; 14(5):396-404.
Fishbein I et al. Bisphosphonate-mediated gene vector delivery from the metal surfaces of stents. Proc Natl Acad Sci USA. 2006 Jan. 3; 103(1):159-64. Epub 2005 Dec. 21.
Fishbein I, et al. Local delivery of gene vectors from bare-metal stents by use of a biodegradable synthetic complex inhibits in-stent restenosis in rat carotid arteries. Circulation. 2008 Apr. 22; 117(16):2096-103. Epub 2008 Apr. 14.
French B A, et al. Direct in vivo gene transfer into porcine myocardium using replication-deficient adenoviral vectors. Circulation. 1994 November; 90(5):2414-24
Flugelman et al. Low level in vivo gene transfer into the arterial wall through a perforated balloon catheter. Circulation. 1992 March; 85(3):1110-7.
Gerszten R E, et al. Adhesion of memory lymphocytes to vascular cell adhesion molecule-1-transduced human vascular endothelial cells under simulated physiological flow conditions in vitro. Circ Res. 1996 December; 79(6):1205-15.
Hedman M et al. Safety and feasibility of catheter-based local intracoronary vascular endothelial growth factor gene transfer in the prevention of postangioplasty and in-stent restenosis and in the treatment of chronic myocardial ischemia: phase II results of the Kuopio Angiogenesis Trial (KAT). Circulation. 2003 Jun. 3; 107(21):2677-83. Epub 2003 May 12.
Johnson T W et al. Stent-based delivery of tissue inhibitor of metalloproteinase-3 adenovirus inhibits neointimal formation in porcine coronary arteries. Arterioscler Thromb Vasc Biol. 2005 April; 25(4):754-9. Epub 2005 Jan. 27.
Klugherz B D et al. Gene delivery from a DNA controlled-release stent in porcine coronary arteries. Nat Biotechnol. 2000 November; 18(11): 1181-4.
Klugherz B D et al. Gene delivery to pig coronary arteries from stents carrying antibody-tethered adenovirus. Hum Gene Ther. 2002 Feb. 10; 13(3):443-54.
Laitinen M et al. Catheter-mediated vascular endothelial growth factor gene transfer to human coronary arteries after angioplasty. Hum Gene Ther. 2000 Jan. 20; 11(2):263-70.
Lee E R, et al. Detailed analysis of structures and formulations of cationic lipids for efficient gene transfer to the lung. Hum Gene Ther. 1996 Sep. 10; 7(14):1701-17.
Li W and Szoka F C Jr. Lipid-based Nanoparticles for Nucleic Acid Delivery. Pharm Res. 2007 Jan. 25.
Liu F, et al. Factors controlling the efficiency of cationic lipid-mediated transfection in vivo via intravenous administration. Gene Ther. 1997 June; 4(6):517-23.
Liu L, Zern M A, Lizarzaburu M E, Nantz M H, Wu J. Poly(cationic lipid)-mediated in vivo gene delivery to mouse liver. Gene Ther. 2003 January; 10(2):180-7. PMID: 127 647
Mazur et al. Coron Artery Dis. 1994 September; 5(9):779-86. Lipofectin-mediated versus adenovirus-mediated gene transfer in vitro and in vivo: comparison of canine and porcine model systems.
Muhs A, et al. Preclinical evaluation of inducible nitric oxide synthase lipoplex gene therapy for inhibition of stent-induced vascular neointimal lesion formation. Hum Gene Ther. 2003 Mar. 1; 14(4):375-83.
Nagrani T, Zaher M, Gaddam S, Jabbour G, Baldari D, Baglini R, Duvvuri S. In-stent thrombosis after 68 months of implantation inspite of continuous dual antiplatelet therapy: a case report. Cases J. 2010 Feb. 23; 3(1):68. [Epub ahead of print]
Nikol S, Engelmann M G, Pelisek J, Fuchs A, Golda A, Shimizu M, Mekkaoui C, Rolland P H. Local perivascular application of low amounts of a plasmid encoding for vascular endothelial growth factor (VEGF165) is efficient for therapeutic angiogenesis in pigs. Acta Physiol Scand. 2002 October; 176(2):151-9
Perlstein I et al. DNA delivery from an intravascular stent with a denatured collagen-polylactic-polyglycolic acid-controlled release coating: mechanisms of enhanced transfection. Gene Ther. 2003 August; 10(17):1420-8.
Pfeiffer T, et al. Lipoplex gene transfer of inducible nitric oxide synthase inhibits the reactive intimal hyperplasia after expanded polytetrafluoroethylene bypass grafting. J Vasc Surg. 2006 May; 43(5):1021-7.
Pfeiffer T, Wallich M, Sandmann W, Schrader J, Giidecke A. Lipoplex gene transfer of inducible nitric oxide synthase inhibits the reactive intimal hyperplasia after expanded polytetrafluoroethylene bypass grafting. J Vasc Surg. 2006 May; 43(5):1021-7. PMID: 16678699 Related Articles
Schulick A H et al. Established immunity precludes adenovirus-mediated gene transfer in rat carotid arteries. Potential for immunosuppression and vector engineering to overcome barriers of immunity J Clin Invest. 1997 Jan. 15; 99(2):209-19.
Sharif F et al. Gene-eluting stents: comparison of adenoviral and adeno-associated viral gene delivery to the blood vessel wall in vivo. Hum Gene Ther. 2006 July; 17(7):741-50.
Sharif F, et al. Gene-eluting stents: adenovirus-mediated delivery of eNOS to the blood vessel wall accelerates re-endothelialization and inhibits restenosis. Mol Ther. 2008 October; 16(10):1674-80. Epub 2008 Aug. 19.
Takahashi A et al. Transgene delivery of plasmid DNA to smooth muscle cells and macrophages from a biostable polymer-coated stent. Gene Ther. 2003 August; 10(17): 1471-8.
Torchilin V P. Multifunctional nanocarriers. Adv Drug Deliv Rev. 2006 Dec. 1; 58(14):1532-55. Epub 2006 Sep. 28. Review.
Walter D H et al. Local gene transfer of phVEGF-2 plasmid by gene-eluting stents: an alternative strategy for inhibition of restenosis. Circulation. 2004 Jul. 6; 110(1):36-45. Epub 2004 Jun. 21.
Wu Y X et al. Stent-based local delivery of therapeutic adenovirus effectively reduces neointimal proliferation in porcine coronarie] Di Yi Jun Yi Da Xue Xue Bao. 2003 December; 23(12):1263-5. Chinese.
Yang J P and Huang L. Overcoming the inhibitory effect of serum on lipofection by increasing the charge ratio of cationic liposome to DNA. Gene Ther. 1997 September; 4(9):950-60.

1. An implantable medical device coated in vitro or coated in situ with a nucleic acid-encapsulating liposome based on a mixture of at least one cationic lipid or cationic polymer and at least one helper lipid.

2. A device of claim 1, wherein the cationic lipid is selected from the group comprising (N-[1-(2,3-dioleyloxy)propyl]-n,n,n-trimethylammonium chloride (DOTMA) and dimethyldioctadecylammonium bromide salt (DDAB),. N-(1-(2,3-Dioleoyloxy)propyl)-N,N,N-trimethylammonium methylsulfate (DOTAP) and 1,2-Dioleoyl-3-Trimethylammonium-Propane (chloride salt) (DMRIE).

3. A device of claim 1 or 2, wherein the helper lipid is selected from the group comprising cholesterol (CHOL), 1-Palmitoyl-2-Oleoyl-sn-Glycero-3-Phosphocholine (POPC) and dioleoylphosphotidylethanolamine (DOPE) and any derivatives of gangliosides, sphingosine, sphingomyelin, prostaglandins, arachidonic acid, synthetic polymers or synthetic compounds suitable of forming particles capable of mammalian cell transfection.

4. A device of any preceding claim wherein nucleic acid-encapsulating liposome is based on a mixture of DDAB and DOPE

5. A device of any preceding claim wherein nucleic acid-encapsulating liposome is based on a mixture of DDAB and Chol/POPC

6. A device of any preceding claim wherein nucleic acid-encapsulating liposome is based on a mixture of DOTMA and DOPE

7. A device of any preceding claim, wherein the amount of cationic lipids or polymer and the amount of helper lipids are in a 1:1 to a 1:5 molar ratio.

8. A device of any of claims 1 to 7 wherein the cationic lipid amount (nmole) to nucleic acid amount (μg) are in a 3:1 to 6:1 ratio.

9. A device of any of claims 1 to 8, wherein the nucleic acid is harboured on a plasmid.

10. A device as claimed in claim 1 wherein the nucleic acid encapsulated by the liposome encodes a gene is selected from the group consisting of NOS (nitric oxide synthase) genes, iNOS (inducible NOS), eNOS (endothelial NOS), nNOS (neuronal NOS) and a phosphomimetic NOS, VEGF (vascular endothelial growth factor), (Tissue inhibitor of metalloproteinase) TIMP I-III, protein kinase G, prostacyclin synthase gene and GAX (an anti-proliferative homeobox gene), or AKT1 (V-akt murine thymoma viral oncogene homolog 1), ANGPT1 (Angiopoietin 1), ANGPT2 (Angiopoietin 2), ANGPTL3 (Angiopoietin-like 3), ANGPTL4 (Angiopoietin-like 4), ANPEP (Aminopeptidase), BAH (Brain-specific angiogenesis inhibitor 1), CCL2 (Chemokine (C—C motif) ligand 2), CCL11 (Chemokine (C—C motif) ligand 11), CDH5 (Cadherin 5), COL18A1 (Collagen, type XVIII, alpha 1), COL4A3 (Collagen, type IV, alpha 3), CSF3 (Colony stimulating factor 3 (granulocyte)), CXCL1 (Chemokine (C—X—C motif) ligand 1), CXCL2 (Chemokine (C—X—C motif) ligand 2), CXCL3 (Chemokine (C—X—C motif) ligand 3), CXCL5 (Chemokine (C—X—C motif) ligand 5), CXCL6 (Chemokine (C—X—C motif) ligand 6), CXCL9 (Chemokine (C—X—C motif) ligand 9), CXCL10 (Chemokine (C—X—C motif) ligand 10), CXCL11 (Chemokine (C—X—C motif) ligand 11), ECGF1 (Endothelial cell growth factor 1 (platelet-derived)), EDG1 (Endothelial differentiation, sphingolipid G-protein-coupled receptor, 1), EFNA1 (Ephrin-A1), EFNA2 (Ephrin-A2), EFNA3 (Ephrin-A3), EFNA5 (Ephrin-A5), EFNB2 (Ephrin-B2), EGF (Epidermal growth factor (beta-urogastrone)), ENG (Endoglin (Osler-Rendu-Weber syndrome 1)), EPAS1 (Endothelial PAS domain protein 1), EPHB4 (EPH receptor B4), EREG (Epiregulin), F2 (Coagulation factor II (thrombin)), FGF1 (Fibroblast growth factor 1 (acidic)), FGF2 (Fibroblast growth factor 2 (basic)), FGF6 (Fibroblast growth factor 6), FGFR3 (Fibroblast growth factor receptor 3), FIGF (C-fos induced growth factor (vascular endothelial growth factor D)), FLT1 (Fms-related tyrosine kinase 1), HAND2 (Heart and neural crest derivatives expressed 2), HGF (Hepatocyte growth factor (hepapoietin A; scatter factor)), HIF1A (Hypoxia-inducible factor 1, alpha subunit), HPSE (Heparanase), ID1 (Inhibitor of DNA binding 1), ID3 (Inhibitor of DNA binding 3), IFNA1 (Interferon, alpha 1), IFNB1 (Interferon, beta 1, fibroblast), IFNG (Interferon, gamma), IGF1 (Insulin-like growth factor 1 (somatomedin C)), IL10 (Interleukin 10), IL12A (Interleukin 12A), IL18 (Interleukin 18 (interferon-gamma-inducing factor)), IL1B (Interleukin 1, beta), IL6 (Interleukin 6 (interferon, beta 2)), IL8 (Interleukin 8), ITGAV (Integrin, alpha V), ITGB3 (Integrin, beta 3), JAG1 (Jagged 1), KDR (Kinase insert domain receptor (a type III receptor tyrosine kinase)), LAMAS (Laminin, alpha 5), LECT1 (Leukocyte cell derived chemotaxin 1), LEP (Leptin (obesity homolog, mouse)), MDK (Midkine (neurite growth-promoting factor 2)), MMP19 (Matrix metallopeptidase 19), MMP2 (Matrix metallopeptidase 2), MMP9 (Matrix metallopeptidase 9), NOTCH4 (Notch homolog 4 (Drosophila)), NPPB (Natriuretic peptide precursor B), NPR1 (Natriuretic peptide receptor A/guanylate cyclase A), NRP1 (Neuropilin 1), NRP2 (Neuropilin 2,), NUDT6 (Nudix (nucleoside diphosphate linked moiety X)-type motif 6), PDGFA (Platelet-derived growth factor alpha polypeptide), PDGFB (Platelet-derived growth factor beta polypeptide), PECAM1 (Platelet/endothelial cell adhesion molecule (CD31 antigen)), PF4 (Platelet factor 4 (chemokine (C—X—C motif) ligand 4)), PGF (Placental growth factor), PLAU (Plasminogen activator, urokinase), PLG (Plasminogen), PLXDC1 (Plexin domain containing 1), PROK2 (Prokineticin 2), PTEN (Phosphatase and tensin homolog), PTGS1 (Prostaglandin-endoperoxide synthase 1), PTGS2 (Prostaglandin-endoperoxide synthase 2), PTN (Pleiotrophin), ANG (Angiogenin, ribonuclease, RNase A family, 5), OPN/SPP1 (Osteopontin), SERPINF1 (Serpin peptidase inhibitor, Glade F, member 1), SH2D2A (SH2 domain protein 2A), SPHK1 (Sphingosine kinase 1), STAB1 (Stabilin 1), STAB2 (Stabilin 2), TEK (TEK tyrosine kinase, endothelial), TGFA (Transforming growth factor, alpha), TGFB1 (Transforming growth factor, beta 1), TGFB2 (Transforming growth factor, beta 2), TGFB3 (Transforming growth factor, beta 3), TGFBR1 (Transforming growth factor, beta receptor I), THBS1 (Thrombospondin 1), THBS2 (Thrombospondin 2), TIE1 (Tyrosine kinase with immunoglobulin-like and EGF-like domains 1), TIMP1 (TIMP metallopeptidase inhibitor 1), TIMP2 (TIMP metallopeptidase inhibitor 2), TIMP3 (TIMP metallopeptidase inhibitor 3), TNF (Tumor necrosis factor), TNFAIP2 (Tumor necrosis factor, alpha-induced protein 2), TNFRSF12A (Tumor necrosis factor receptor superfamily, member 12A), TNFSF15 (Tumor necrosis factor (ligand) superfamily, member 15), TNNT1 (Troponin T type 1), VEGF (Vascular endothelial growth factor), VEGFB (Vascular endothelial growth factor B), VEGFC (Vascular endothelial growth factor C), AGGF1 (Angiogenic factor with G patch and FHA domains 1).

11. A device as claimed in claim 10 wherein the nucleic acid encapsulated by the liposome encodes the gene for eNOS (endothelial NOS) and IL10 (Interleukin 10).

12. A device as claimed in any preceding claim wherein the device is phosphatidylcholine-coated or phosphatidylcholine derivative-coated.

13. A device as claimed in any preceding claim wherein the device is selected from a stent, a coronary or peripheral artery stent, intravenous canula, urinary catheter, implantable coronary device, cardiac patch, bone plate, bone prosthesis, bone patch, dental implant, blood vessel prosthesis, artificial heart valve, skin repair device, heart patch, bone patch, or contact lenss.

14. A method of producing a nucleic acid-encapsulating liposome-coated device comprising:—
(a) mixing a nucleic acid or peptide of interest with a mixture of any cationic lipid or polymer and a “helper lipids” or lipids to form a lipoplexe/liposome, and
(b) coating the device with the lipoplex.

15. A method as claimed in claim 14 wherein the stent is coated in vitro.

16. A method as claimed in claim 14 wherein the stent is coated in vivo.

17. A method of any of claims 14 to 16, wherein the step (a) comprises mixing a nucleic acid or peptide of interest with a mixture of DDAB and DOPE to form a lipoplex.

18. A method of any of claims 14 to 16 wherein the step (a) comprises, mixing a nucleic acid or peptide of interest with a mixture of DDAB and Chol/POPC to form a lipoplex.

19. A method of any of claims 14 to 16 wherein the step (a) comprises mixing a nucleic acid or peptide of interest with a mixture of DOTMA and DOPE to form a lipoplex.

20. A method of delivering a gene to a site in the body comprising use of a device as claimed in any of claims 1 to 13.

21. A device of claims 1 to 13, or the method of claims 14 to 20, wherein the device is a stent.

22. A method of prevention of stenosis or restenosis comprising use of a liposome-coated stent of claim 13.
